(19) United States
(12) Patent Application Publication
(10) Pub. No.: US 2012/0232274 A1
(43) Pub. Date: Sep. 13, 2012

(54) PROCESS FOR PREPARATION OF CLOPIODOGREL BISULFATE FORM-1

(75) Inventors: Wolfgang Burger, Raubling (DE); Omprakash Gudaparthi, Hyderabad (IN); Konda Srinivasa Reddy, Gorijavoluguntapalem (Village) (IN); Suneel K. Kumar, Kakinada (IN); Venkata P. Ramana, Visakhapatnam (IN)

(73) Assignee: PHARMAZELL GMBH, Raubling (DE)

(21) Appl. No.: 13/508,545

(22) PCT Filed: Mar. 22, 2010
(86) PCT No.: PCT/IN2010/000171

§ 371 Date: May 8, 2012

(30) Foreign Application Priority Data
Nov. 9, 2009 (IN) 2717/CHE/2009

(51) Int. Cl. C07D 495/04

(52) U.S. Cl. 546/114

(57) ABSTRACT

An improved process for preparing crystalline form-1 of (S)-methyl 2-(2-chlorophenyl)-2-{6,7-dihydrothieno[3,2-c]pyridine-5(4H)-yl}acetate bisulfate (clopidogrel bisulfate) of formula I is provided The preparation comprises the straight conversion of an uncyclized material of (S)-methyl 2-[2-(thiophen-2-yl)ethylamino]-2-(2-chlorophenyl) acetate hydrochloride into clopidogrel bisulfate crystalline form-1 without any degradation of clopidogrel base

### FIELD OF INVENTION

The present invention relates to an improved process for the preparation of Clopidogrel bisulfate crystalline form-1 of formula I from (S)-methyl-2-(2-thiophen-2-yl) ethylamino)-2-(2-chlorophenyl) acetate hydrochloride of formula II. The present invention also provides a highly pure crystalline form of Clopidogrel bisulfate from (S)-methyl-2-(2-thiophen-2-yl) ethylamino)-2-(2-chlorophenyl) acetate hydrochloride of formula II without any degradation of Clopidogrel base.

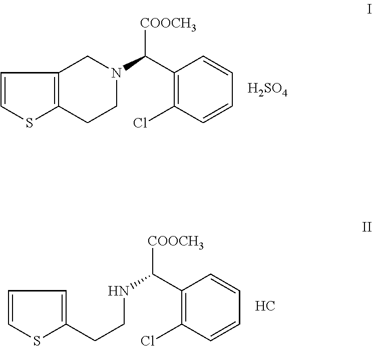

### BACKGROUND OF INVENTION

(S)-(+)-(2-chlorophenyl)-2-(6,7-dihydro4H-thieno[3,2c]pyridine-5-yl-acetate hydrogen sulfate, known for platelet aggregation inhibitor, drug having International Nonproprietary Name [INN] Clopidogrel hydrogensulfate.

Clopidogrel is administrated as it bisulfate salt and currently being marketed under the brand name PLAVIX™.

Clopidogrel hydrogen sulfate was first revealed in U.S. Pat. No. 4,847,265 assigned to Sanofi SA and was claimed as dextrorotatory isomer of methyl α-5(4,5,6,7-tetrahydro (3,2-c) thieno pyridyl) (2-chlorophenyl)-acetate and its salts. The separation of enantiomers [dextrorotatory enantiomers and leavo rotatory enantiomers] from the racemic mixture of methyl α-5(4,5,6,7-tetrahydro(3,2-c)thieno pyridyl) (2-chlorophenyl)-acetate in the specification as illustrated in Scheme-1 of U.S. Pat. No. 4,847,265 (as given below)

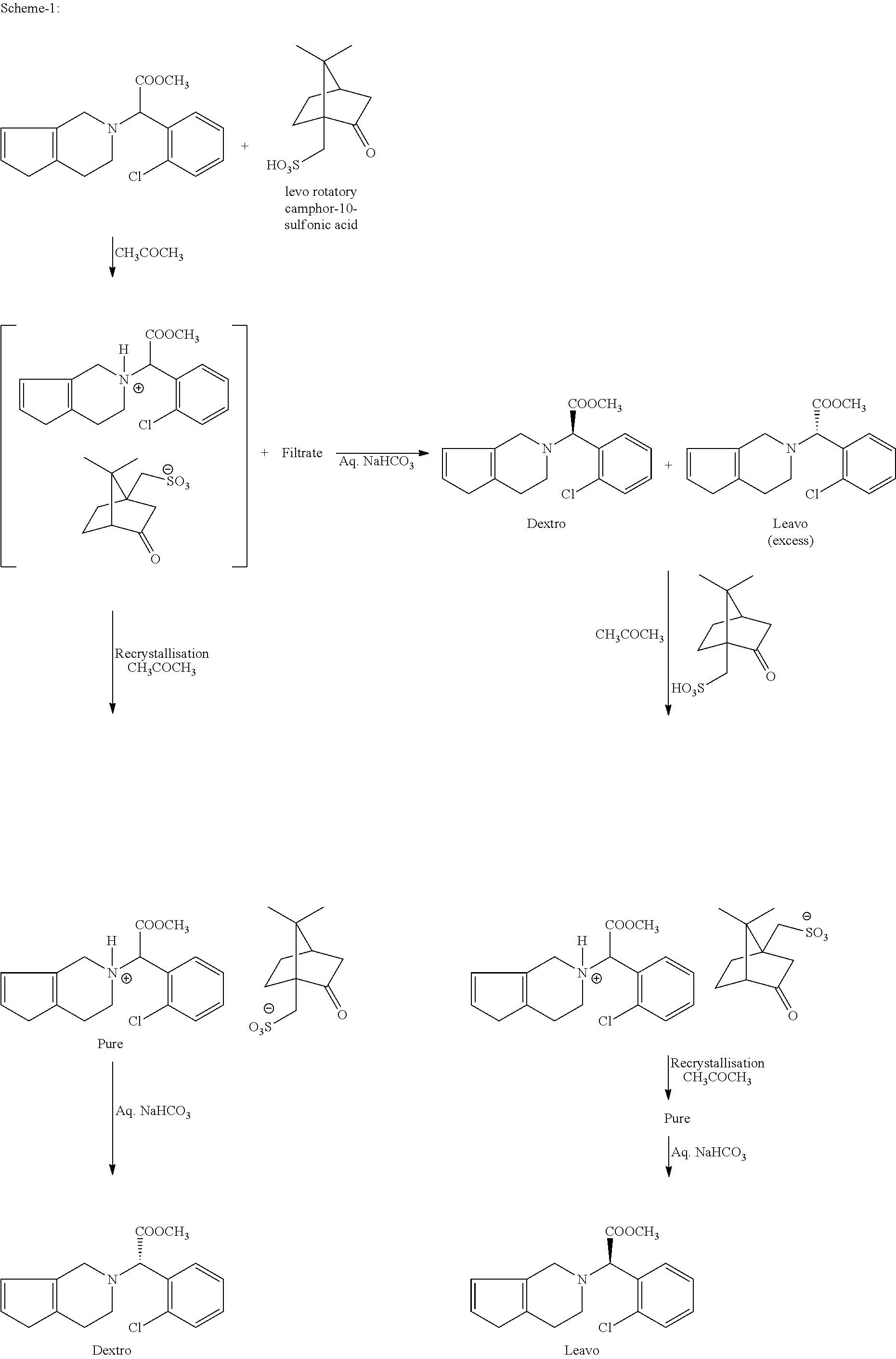

The product is characterized by its melting point and optical rotation, which are 182° C. and [α]_{D}²⁰=+51.61 [c=2.044 g/100 ml, methanol] respectively. The specification does not deal with the crystalline form of Clopidogrel hydrogen sulphate prepared in this way.

EP99802 provides a new process for the preparation of Clopidogrel bisulfate. JP4230387 also discloses the process for the preparation of (+)-Clopidogrel bisulfate in detail with a few modifications.

The polymorphic forms 1 and 2 for Clopidogrel bisulfate were first revealed in U.S. Pat. No. 6,504,030 assigned to Sanofi. It reveals that Polymorphic form-1 was prepared according to the method described in U.S. Pat. No. 4,847,265. Polymorphic form-1 is specified as monocline crystal form, characterized by X-ray Diffraction pattern and Infrared spectrum.

Melting point and Optical rotation of polymorph form are 184° C. and [α]_{D}²⁰=+55.10 [c=1.891/100 ml, methanol], respectively. Its melting point of 176° C. characterizes the orthorhombic polymorph form 2.

The study of polymorphic forms 1, 2, 3, 4, 5 and its preparations are extensively revealed in U.S. Pat. No. 7,074,928 assigned to Teva Pharmaceutical Industries Ltd.

U.S. Pat. No. 6,429,210 discloses that Form 2 exhibits a lower solubility than Form 1 as a result of its greater thermodynamic stability

U.S. Pat. No. 7,291,735 discloses a process for the preparation of blood-platelet aggregation inhibiting agent, in particular Methyl-(+)-(S)-.alpha.-(2-chlorophenyl)-6,7-dihydrothieno [3,2-c]pyridine-S-(4H)acetate bisulfate Form-1. It also reveals the preparation of 99.96% pure (+)(S)-Clopidogrel from the racemic mixture of Clopidogrel base. The obtained (+)(S)-Clopidogrel is dissolved in ethyl acetate and treated with Concentrated sulphuric acid followed by the seeding with Clopidogrel bisulfate form-1 to prepare (+) (S)-Clopidogrel bisulfate crystals.

US Patent Application 2006047121 reveals a process for the preparation of Clopidogrel bisulfate form-1 from form-2. It reveals that form-1 was prepared by dissolving form-2 in C₁₋₄ carboxylic acid followed by the addition of antisolvent i.e. dimethyl ether; diethyl ether; and diisopropyl ether.

European patent 1554284 reveals the process for the preparation of hydrogen sulfate (α-S) of α-(2-chlorophenyl)-6,7-dihydro-thieno[3,2c]pyridine-5(4H)-acetic acid methyl ester in crystalline form 1, which comprise the separating out of a solution of Clopidogrel in form of a free base or salt in a solvent selected form primary, secondary or tertiary C₁₋₅ alcohols or their ester with C₁₋₄ carboxylic acids or optionally their mixture.

PCT Application 2008118030 reveals a process for the preparation of substantially pure Clopidogrel Bisulfate form-1. It reveals the preparation of form-1 by treating sulfuric acid with optically active Clopidogrel base in the presence of mixture of at least two solvents. The first one chosen from group I, comprising aliphatic ethers, and the second one from group II, comprising ketones, esters of C₁₋₅ carboxylic acids and C₁₋₄ aliphatic alcohols, primary, secondary and tertiary aliphatic C₁₋₄ alcohols.

US Patent Application 2009247569 reveals a process for the preparation of Clopidogrel Bisulphate form-1 comprising, dissolving Clopidogrel base in an organic solvent like C₆ ketone, C₆₋₁₂ aromatic hydrocarbon to obtain the solution; and addition the sulfuric acid to the solution. It also reveals a novel process for the preparation of. Form-1, comprises dissolving Clopidogrel base in MTBE (methyl-t-butyl-ether), cooling, adding formic acid or acetic acid to obtain a cooled solution; and adding the cooled solution to a mixture of sulfuric acid and MTBE (methyl-t-butyl-ether) at a temperature less than about 40° C.

The above-mentioned prior-art methods are inconsistent in the presence Form2, which is one of the major impurity. So, there exists a need for still further improvement of the economical process for the production of Clopidogrel Bisulfate, with high purity and without detectable minimized polymorphic impurities specifically Form-2.

The present invention provides a novel and industrially feasible process for the preparation of form-1 crystalline form of Clopidogrel hydrogen sulfate minimized polymorphic impurities and the crystalline form-2.

### SUMMARY OF THE INVENTION

The present invention reveals the novel process for the preparation of Clopidogrel bisulfate in crystalline form-1 from (S)-methyl-2-(2-thiophen-2-yl) ethylamino)-2-(2-chlorophenyl) acetate hydrochloride without any degradation of Clopidogrel base.

Clopidogrel bisulfate crystalline form-1 obtained in the current process is of highly pure.

In one embodiment of the present invention Clopidogrel base is not isolated and is directly obtained as form-1.

In yet another embodiment, present invention is useful to control the OVI [Organic Volatile Impurities] impurities as per the guidelines of ICH [International Conference on Harmonisation of Technical Requirements for Registration of Pharmaceuticals for Human Use].

### DETAILED DESCRIPTION OF THE INVENTION

The present invention relates to an improved process for the preparation of Clopidogrel Bisulfate crystalline Form-1 of formula from (S)-methyl-2-(2-thieno-2-yl)ethylamino)-2-(2-chlorophenyl) acetate hydrochloride of formula II. The present invention also provides a highly pure crystalline form of Clopidogrel bisulfate from (S)-methyl-2-(2-thieno-2-yl)ethylamino)-2-(2-chlorophenyl) acetate hydrochloride of formula II without any degradation of Clopidogrel base.

The improved process for the preparation of Clopidogrel bisulfate form-1 of formula (I) comprises steps of:

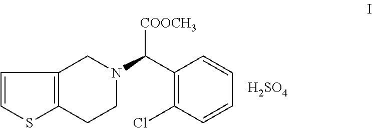

i. (S)-methyl-2-(2-thiophen-2-yl)ethylamino)-2-(2-chlorophenyl) acetate hydrochloride of general formula II is treated with 37-41% WN formaldehyde solution at a temperature of 25-30° C. and then to a temperature raised to 50-55° C.

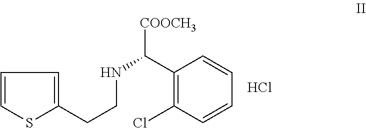

ii. The reaction is then cooled to a temperature of 25-30° C. and is continued until (S)-methyl-2-(2-thiophen-2-yl)ethylamino)-2-(2-chlorophenyl) acetate hydrochloride content reaches to <0.5%. The reaction is then cooled to a temperature range of 5-10° C.
iii. C₁₋₄ alcohol, C₁₋₅ carboxylic acid ester are added to the reaction and then pH is adjusted to a range of 7-8 by employing a base. The reaction is heated to a temperature of 25-30° C.
iv. Aqueous layer and organic layers are separated and aqueous layer is further extracted with C₁₋₅ carboxylic acid ester.
v. Combined C₁₋₅ carboxylic acid ester layer is washed with 1-20% sulphuric acid solution to remove the impurities.
vi. Then C₁₋₅ carboxylic acid ester layer is washed with inorganic base solution followed by water.
vii. The organic layer obtained is treated with activated charcoal at a temperature of 25-30° C. for about 20-30 minutes. The reaction mass is then filtered. viii. Sulfuric acid dissolved in C₁₋₅ carboxylic acid ester is added to the filtered reaction mass at a temperature of −10-0° C. for about 90-120 minutes.
ix. C₁₋₅ carboxylic acid is added slowly to the reaction mass for 30-45 mins at the same temperature. The reaction mass is heated to a temperature of 25-30° C. and is maintained for 20-24 hrs.
The precipitated solid is filtered, washed with C₁₋₅ carboxylic acid ester; the suck dried cake is washed with Acetone.
x. The crystalline material obtained is dried at 40-45° C. under vacuum until LOD and OVI reaches as per limit.
   Schematically the present process can be represented as:

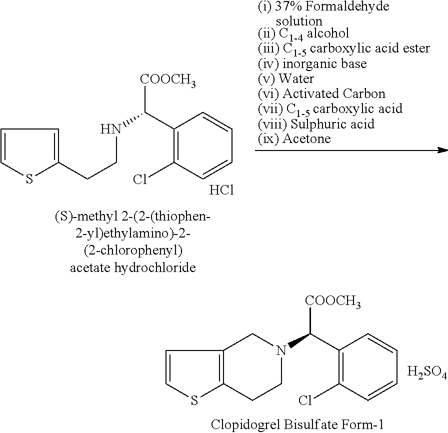

(S)-methyl-2-(2-thiophen-2-yl)ethylamino)-2-(2-chlorophenyl) acetate hydrochloride (obtained from known methods of prior art), formaldehyde employed in step (i) are 1 w/w and 8 V/W respectively. C₁₄ alcohol employed in step (iii) is selected from the group of simple acyclic alcohol, preferably methanol. C₁₋₅ carboxylic acid ester is ethyl acetate, n-butyl acetate etc., preferably n-butyl acetate.

Inorganic base employed in step (vi) is alkali metal carbonates or bicarbonates, preferably sodium bicarbonate, more preferably 5% sodium bicarbonate and C₁₋₅ carboxylic acid employed in step (ix) is acetic acid

Sulfuric acids, C1-5 carboxylic acid employed in step (viii), step (ix) are 1 mole equivalent of the reaction mass.

pH of the solution in step (vi) i.e. after using inorganic base is in the range from 7 to 8.

Employing C1-5 carboxylic acid esters in step (iii) will provide the improved quality of the final product.

Employing C₁₋₄ alcohol in step (iii) is providing the improved quality, particularly control of the other surplus isomer along with three mysterious impurities.

Washing of C₁₋₅ carboxylic acid ester layer with 1% sulphuric acid solution to remove the major tricky mysterious impurities excluding the methanol controlled.

After the acidic wash pH of the reaction mass is adjusted to a range of 7-8 by employing inorganic base wash followed by water wash. If the above operation is not conducted during the formation of Form-1, infrequently no solid separation and even the separated content will also be gummy in nature.

Subsequently, water is removed from the organic layer by applying high vacuum 1-2 mbar at a temperature range of 30-40° C. This will provide the best results for water removal and the usage of drying agents can also be avoided.

The above step is very crucial, without the water removal operation further proceeding into the reaction leads to Form-2.

A high volume of C₁₋₅ carboxylic acid ester is preferable to avoid the formation of form-2. And employing C₁₋₅ carboxylic acid also avoids the formation of form-2.

Cyclohexane washing in the final stage while preparing form-1 of Clopidogrel bisulfate is useful for controlling the OVI impurities as per the guidelines of ICH.

### EXAMPLES

### Example—1

### Clopidogrel Bisulfate Form-1

(I) (S)-methyl-2-(2-thiophen-2-yl)ethylamino)-2-(2-chlorophenyl) acetate hydrochloride is treated with 37-41% W/V formaldehyde solution at a temperature of 25-30° C. and then slowly heated to a temperature of 50-55° C. and continued until (S)-methyl-2-(2-thiophen-2-yl) ethylamino)-2-(2-chlorophenyl)acetate hydrochloride content reaches to <0.5%. The reaction is then cooled to a temperature range of 5-10° C. Methanol, n-butyl acetate are added to the reaction and then pH is adjusted to a range of 7-8 by employing a base. The reaction is heated to a temperature of 25-30° C. Aqueous layer of reaction mass is further extracted with n-butyl acetate and then the layers are combined. n-Butyl acetate layer is washed with 1% sulphuric acid solution to remove the impurities. Now, the n-butyl acetate layer is washed with 5% sodium bicarbonate solution followed by water. Distilled off about 10% of the n-butyl acetate under vacuum till the moisture content is below 0.5%. The pre-dried Activated Charcoal is added to the organic layer at a temperature of 25-30° C. for 20-30 minutes. The reaction mass is then filtered for making particle free.

(II) Sulfuric acid dissolved in n-butyl acetate is added to the filtered reaction mass at a temperature of −10-0° C. for 90-120 minutes. Acetic acid is added slowly to the reaction mass for 30-45 minutes at the same temperature. The reaction mass is heated to a temperature of 25-30° C. and is maintained for 20-24 hours. The gummy free material is filtered, washed with n-butyl acetate, kept under suck dry for 30-60 minutes. Now, the cake is washed with acetone and kept under suck dry for 30-60 minutes. The obtained crystalline material is dried at 40-45° C. under vacuum until LOD and OVI reaches as per limit.

We claim:

1. An improved process for the preparation of Clopidogrel bisulfate of general formula I comprising steps of: 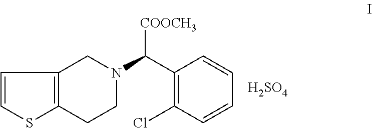
i. Treating (S)-methyl-2-(2-thiophen-2-yl) ethylamino)-2-(2-chlorophenyl) acetate hydrochloride of general formula II with 37-41% WN formaldehyde solution at a temperature of about 25-30° C. and then heated to a temperature of about 60-65° C., preferably in the range of 50-55° C. 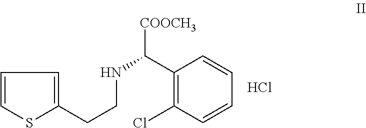
ii. Cooling the reaction mass to a temperature of about 25-30° C. and is continued until (S)-methyl-2-(2-thiophen-2-yl) ethylamino)-2-(2-chlorophenyl) acetate hydrochloride content reaches to <0.5%. The reaction is then cooled to a temperature range of 5-10° C.
iii. Diluting the reaction mass with C₁₄ alcohol, C₁₋₅ carboxylic acid ester and then pH is adjusted to a range of about 7-8 by employing a base. The reaction mass is allowed to reach to a temperature of about 25-30° C.
iv. Separating the aqueous and organic layers obtained from step (iii) and aqueous layer is further extracted with C₁₋₅ carboxylic acid ester and then the organic layers are combined.
v. Washing the combined organic layer with 1-20% sulphuric acid solution to remove the impurities.
vi. Washing the combined layer with inorganic base solution followed by water.
vii. Treating the organic layer with activated charcoal at a temperature of about 25-30° C. for about 20-30 minutes. The treated layer obtained is filtered to provide a filtered solution.
viii. Adding sulfuric acid dissolved in C₁₋₅ carboxylic acid ester to the filtered solution at a temperature of about −10-0° C. for 90-120 minutes.
ix. Adding C₁₋₅ carboxylic acid is added slowly to the solution obtained in step (viii) for about 30-45 minutes at the same temperature and then temperature is raised to about 25-30° C. and is maintained for about 20-24 hrs.
x. Filtering the solid material obtained from step (xi), and washing with C₁₋₅ carboxylic acid ester, acetone and kept under suck dry for 30-60 minutes to provide a crystalline solid material.
xi. The crystalline solid material obtained is dried at about 50-55° C., preferably at 40-45° C. under vacuum.

2. The process as claimed in claim 1, (S)-methyl-2-(2-thiophen-2-yl)ethylamino)-2-(2-chlorophenyl) acetate hydrochloride, formaldehyde employed in step (i) are 1 w/w and 8 WAV respectively.

3. The process as claimed in claim 1, C₁₋₄ alcohol employed in step (iii) is selected from the group of simple acyclic alcohol, preferably methanol.

4. The process as claimed in claim 1, C₁₋₅ carboxylic acid ester is ethyl acetate, n-butyl acetate etc., preferably n-butyl acetate.

5. The process as claimed in claim 1, inorganic base employed in step (vi) is alkali metal carbonates or bicarbonates, preferably sodium bicarbonate, more preferably 5% sodium bicarbonate.

6. The process as claimed in claim 1, sulfuric acid, C₁₋₅ carboxylic acid employed in step (viii), step (ix) are 1 mole equivalent of the reaction mass.

7. The process as claimed in claim 1, claim 6, C₁₋₅ carboxylic acid employed in step (ix) is acetic acid.

8. The process as claimed in claim 1, pH of the solution in step (vi) is in the range from 7 to 8.

9. The process as claimed in claim 1, the usage of methanol in step (iii) is to control the surplus isomer along with the impurities.
